(19) United States
(12) Patent Application Publication
(10) Pub. No.: US 2016/0051506 A1
(43) Pub. Date: Feb. 25, 2016

(54) Fat Emulsion for Artificially Feeding Seriously Ill Intensive Care Patients

(71) Applicant: B. Braun Melsungen AG, Melsung (DE)

(72) Inventor: Michael Boll, Melsungen (DE)

(21) Appl. No.: 14/930,723

(22) Filed: Nov. 3, 2015

Related U.S. Application Data
(63) Continuation of application No. 13/126,245, filed on Apr. 27, 2011, now abandoned.

(30) Foreign Application Priority Data
Nov. 18, 2008 (DE) 102008057867.3

(51) Int. Cl. A61K 31/225; A23L 1/30; A61K 9/107

(52) U.S. Cl. CPC A61K 31/225; A61K 9/107; A23L 1/3008; A23V 2002/00

(57) ABSTRACT

The present invention relates to a pharmaceutical formulation for the prophylaxis and treatment of critical illness polyneuropathy (CIP) and critical illness myopathy (CIM). The invention further relates to an isotonic fat emulsion comprising at least one triglyceride that comprises at least one fatty acid group having an odd number of carbon atoms, wherein the fatty acid group comprises a carbon chain having 5 to 15 carbon atoms.

The present invention relates to a pharmaceutical formulation for the prophylaxis and treatment of critical illness polyneuropathy (CIP) and critical illness myopathy (CIM). Further, the invention relates to an isotonic fat emulsion comprising at least one triglyceride including at least one fatty add residue with an odd number of carbon atoms, wherein said fatty acid residue includes a carbon chain with from 5 to 15 carbon atoms. In addition, the invention relates to the use of the isotonic fat emulsion as a dietary product, and the invention further relates to the use of the pharmaceutical formulation/isotonic fat emulsion within the scope of parenteral nutrition or as a component of a dietary product, especially the use of a fat emulsion for artificially feeding septic intensive care patients.

Due to the progress in intensive care medicine of recent years and decades, which has been due to among others novel treatment concepts, classification systems and well-aimed interventions, the survival times of extremely ill intensive care patients could be prolonged significantly. However, consequently, clinical pictures that were previously rare or unknown have been observed in this group of patients. Thus, intensive care patients have a particularly high risk of developing a sepsis, which may entail serious complications in the further course thereof. Among these, critical illness polyneuropathy (CIP) and critical illness myopathy (CIM) were determined in systematic studies on neurological and muscular problems in intensive care patients. Both cases involve acquired muscle weakness, namely CIP, which is primarily axonal, and CIM, which is primarily muscular. A clinical delimitation of CIP from CIM is extremely difficult, since generalized paralysis, myasthenia and respirator weaning problems are the most important and common symptoms in both cases, and in addition, both diseases can occur together (O. Friedrich, E. Hund, Anaesthesist 2006, 55, 1271-1280). For the prevalence of CIP/CIM, a value of 70-80% is currently stated for patients with severe sepsis and multiple organ failure. Although there is probably not a higher lethality due to CIP/CIM alone, the occurrence of CIP/CIM prolongs the intensive care therapy, delays the rehabilitation and thereby leads to an enormous increase of the treatment and macroeconomic costs. In addition, muscular weakness and rapid exhaustion of patients who are suffering from severe ARDS, for example, is considered the most important cause of limited quality of life even after 12 months from the end of the intensive care medical treatment (M. S. Herridge, A. M. Cheung, C. M. Tansey, N. Engl. 1 Med. 2003, 348, 683-693). The exact causes of CIP/CIM are currently still unclear and under research in intensive care medicine. Inflammation mediators, catabolism, insulin resistance, the application of corticosteroids, increased glucagon sensitivity, energy supply disorder, the application of muscle relaxants, oxidative stress as well as general microcirculatory and/or inflammation reactions among others are discussed as risk factors. For this reason, specific therapies for CIP/CIM are still unknown. From the literature, it can be seen that many authors consider the aggressive therapy of SIRS and sepsis (“early-goal directed therapy”) as the most important component of a CIP/CIM therapy. Further, an intensified insulin therapy could decrease the incidence by 44%, and another therapeutic option is seen in the intravenous administration of immunoglobulins (M. Alb, S. Hirner, T. Luecke, Anasthesiol. Intensivmed. Notfallmed. Schmerzther. 2007, 4, 250-258).

Another background of the present invention is in the field of artificial feeding of intensive care patients by fat emulsions for intravenous application or by lipid-containing dietary products.

Fat emulsion for parenteral nutrition serve for supplying fats in an intravenously acceptable dosage form if normal oral feeding is not possible or medically contraindicated. Fat emulsions common in the prior art are prepared from vegetable oils, such as safflower oil or soybean oil; in some cases, they additionally contain triglycerides of medium-chain fatty acids (so-called medium-chain triglycerides (MCT)) and/or oils of marine origin (fish oils, mostly from cold-water fish).

Thus, DE-OS-37 21 137 describes lipid emulsions for parenteral nutrition comprising eicosapentaenic acid triglyceride and/or docosahexaenic acid triglyceride, or fish oils containing such triglycerides, as well as vegetable oils containing omega-6 fatty acids, and MCT.

EP 0 120 169 B1 discloses synthetic triglycerides which may bear a polyunsaturated fatty acid (preferably eicosapentaenic acid) at the central carbon atom of the glycerol molecule. The glycerides prepared according to this definition may be used for nutrition, as a food supplement or medicament for therapeutic nutrition.

U.S. Pat. No. 4,526,902 describes mixtures comprising 25-75% by weight of eicosapentaenic acid and an omega-6 fatty acid that are used as a component of pharmaceuticals or fat-containing foods, such as butter or the like.

U.S. Pat. No. 6,740,679 describes n-heptanoic acid as an energy source for patients suffering from disorders of the degradation of long-chain fatty acids.

US 2008/0132571 A1 discloses formulations and methods for the treatment of catabolic effects in patients, wherein odd-numbered fatty acids and their glycerides are applied for enhancing the intracellular ratio of AMP to ATP and for enhancing the activity of AMP-activated protein kinase (AMPK).

Effective treatment methods for CIP and CIM are hardly known. In addition, effective nutritive methods for treating sepsis and the secondary complications of intensive care therapy, such as CIP and/or CIM, are hardly known to date.

Thus, there is a need for substances and formulations for artificial feeding and the accompanying nutritive treatment of intensive care patients, and there is an urgent need for formulations and methods for the prophylaxis and therapy of CIP and/or CIM.

Before this background, the object of the present invention is to provide a pharmaceutical formulation for the accompanying nutritive treatment of critically ill, for example, septic, intensive care patients and for the prophylaxis and therapy of secondary complications of intensive care therapy, such as CIP and/or CIM.

Surprisingly, it has been found that the frequency of the occurrence of the mentioned complications can be reduced, or the severity of the disease alleviated, or its course shortened, by supplying a fat emulsion containing triglycerides with fatty acid residues having an odd number of carbon atoms.

Thus, the present invention relates to a pharmaceutical formulation for the prophylaxis or treatment of CIP and/or CIM comprising a fat emulsion containing at least one triglyceride (A) of formula (I):

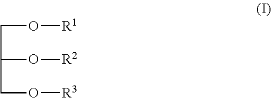

wherein at least one of radicals R¹, R² or R³ is independently an alkanoyl radical having an odd number of from 5 to 15 carbon atoms.

The triglyceride (A) of the fat emulsion to be used according to the invention consists of glycerol esterified with fatty acids at least one of which has an odd number of carbon atoms with from 5 to 15 carbon atoms.

According to the present invention, the odd-numbered alkanoyls have a chain length of from 5 to 15 carbon atoms. Preferably, they are alkanoyls derived from one or more of the following fatty acids selected from the group consisting of pentanoic acid (C5:0, n-valeric acid), heptanoic acid (C7:0, enanthic acid), nonanoic acid (C9:0, pelargonic acid), undecanoic acid (C11:0, undecylic acid), tridecanoic acid (C13:0, tridecylic acid) and pentadecanoic acid (C15:0, pentadecylic acid).

Preferably, at least one of radicals R¹, R² or R³ in triglyceride (A) independently has a chain length of from 5 to 9 carbon atoms.

More preferably, at least one of radicals R¹, R² or R³ in triglyceride (A) is independently n-heptanoyl, i.e., a triglyceride consisting of esterified glycerol in which at least one, preferably at least two, hydroxy groups are esterified with heptanoic acid.

In particular, triglyceride (A) is triheptanoin, i.e., glycerol in which the three hydroxy groups are esterified with n-heptanoic acid.

The synthesis of triglyceride (A) is familiar to the skilled person. The required odd-numbered fatty acids (pentanoic, heptanoic, nonanoic, undecanoic, tridecanoic and pentadecanoic acids) are commercially available, for example, from Sigma Chemicals Co. Also, there are numerous commercial supply sources of different variants of triglyceride (A) as such: For example, triheptanoin can be purchased from the Condea Chemie GmbH (Witten, Germany) as Special Oil 107. Trinonanoin, triundecanoin or tripentadecanoin can be supplied, for example, by the Chemos GmbH (Regenstauf, Germany).

In addition to the essential fatty acid residues having an odd carbon number of from 5 to 15 according to the invention, the triglyceride (A) may also contain other, even-numbered fatty acid residues. In this case, fat emulsions containing triglyceride (A) in which at least one fatty acid residue is derived from omega-3 and/or omega-6 fatty acids are preferred in the pharmaceutical formulation according to the invention. Omega-3 and omega-6 fatty acids are biologically essential building blocks/nutrients which the human organism itself cannot produce completely and which function as precursors for prostaglandins, eicosanoids and structural components of cell membranes. Various oils of vegetable origin including soybean oil and safflower oil serve as a source of omega-6 fatty acids; their use for the preparation of intravenous fat emulsions is included in the prior art. Also included in the prior art is the processing of oils of marine origin (“fish oil”) as a source of omega-3 fatty acids in intravenous fat emulsions (EP-A-0 298 293), wherein highly purified fish oil concentrates are preferred, which are obtained from cold-water fish, such as salmons, herrings, sardines or mackerels. Their content of omega-3 fatty acids is preferably 40% or more.

Further preferred is triglyceride (A) in which at least one fatty acid residue is selected from the group consisting of medium-chain fatty acids (e.g., caprylic acid C8:0, capric acid C10:0, lauric acid C12:0), long-chain saturated fatty acids (e.g., myristic acid C14:0, palmitic acid C16:0, stearic acid C18:0), monounsaturated fatty acids (palmitoleic acid C16:1, oleic acid C18:1), polyunsaturated fatty acids of omega-3 and omega-6 type, for example, eicosapentaenic acid (EPA, C20:5 omega-3), docosahexaenic acid (DHA, C22:6 omega-3), linolic acid (LA, C18:2 omega-6) or gamma-linolenic acid (GLA, C18:3 omega-6).

In a further preferred embodiment of the present invention, triglyceride (A) is in the form of a randomized structured lipid with a random distribution of the alkanoyl residues with an odd carbon number and alkanoyl residues with an even carbon number in positions sn-1, sn-2 and sn-3 of the triglyceride molecule (A). Alternatively and especially preferably, triglyceride (A) is in the form of a chemically defined structured lipid, i.e., there is a chemically defined distribution of the alkanoyl residues with an odd carbon number in positions sn-1 and sn-3 and alkanoyl residues with an even carbon number in position sn-2 of the triglyceride molecule.

In the case where the fat emulsions to be employed according to the invention wholly of in part contain chemically defined or randomized structured lipids, vegetable oils preferably serve as a source of omega-6 fatty acids, and fish oils serves as a source of omega-3 fatty acids for the preparation of the structured lipids. Randomized structured triglycerides are obtainable, for example, by the chemical transesterification of a mixture of a desired vegetable and/or fish oil with a triglyceride consisting of odd-numbered fatty acids having a chain length of from 5 to 15. In the case of chemically defined structured lipids, the transesterification is effected enzymatically from the same base materials.

In a further preferred embodiment of the present invention, the pharmaceutical formulation according to the invention comprises a fat emulsion that includes at least one additional triglyceride (B) different from (A).

Preferably, triglyceride (B) includes triglycerides of marine or vegetable origin. Since the recovery of pure omega-3 or omega-6 fatty acids from fish oils or vegetable oils or the chemical synthesis of these fatty acids is tedious and costly on the one hand, while the mentioned oils of marine or vegetable origin have a high content of the corresponding fatty acids on the other, it is not required according to the present invention to isolate omega-3 or omega-6 fatty acids or triglycerides containing omega-3 or omega-6 fatty acids from these oils, but the oils can be used as such for the preparation of the fat emulsions to be employed according to the invention.

The use of fish oil and especially that of soybean or safflower oil automatically provides long-chain saturated fatty acids as well, such as the representatives myristic, palmitic and stearic acids as mentioned above, and also oleic acid, which is monounsaturated and contained in both marine oils and, in an especially high concentration, in olive oil.

Medium-chain fatty acids or triglycerides are contained in semisynthetic MCT oil (Miglyol oil), i.e., at more than 90% (based on the total fatty acid content) as caprylic and caprinic acids. In this form, they are particularly suitable to be employed as triglyceride (B) in the fat emulsion to be employed according to the invention.

In a preferred embodiment, the pharmaceutical formulation according to the invention includes an emulsion that comprises, in addition to triglyceride (A), at least one other triglyceride (B) containing at least one fatty acid residue selected from the group consisting of medium-chain fatty acids (e.g., caprylic acid C8:0, capric acid C10:0, lauric acid C12:0), long-chain saturated fatty acids (e.g., myristic acid C14:0, palmitic acid C16:0, stearic acid C18:0), monounsaturated fatty acids (palmitoleic acid C16:1, oleic acid C18:1), polyunsaturated fatty acids of omega-3 and omega-6 type, for example, eicosapentaenic acid (EPA, C20:5 omega-3), docosahexaenic acid (DHA, C22:6 omega-3), linolic acid (LA, C18:2 omega-6) or gamma-linolenic acid (GLA, C18:3 omega-6).

In a further preferred embodiment of the present invention, the amount of triglyceride (A) is from 50 to 80% by weight, more preferably from 60 to 70% by weight, based on the total weight of all triglycerides in the emulsion.

Preferably, the pharmaceutical formulation according to the invention includes triglycerides in an amount of from 5 to 30% by weight, more preferably from 10 to 20% by weight, based on the total pharmaceutical formulation.

In addition to triglyceride (A) and optionally other triglycerides/lipids, the fat emulsion of the pharmaceutical formulation according to the invention advantageously contains water for injection as well as further auxiliaries and additives corresponding to the state of the art in the preparation of intravenous fat emulsions, for example, emulsifiers, co-emulsifiers, stabilizers and suitable substances for adjusting isotonicity.

Physiologically well-tolerated emulsifiers, such as phospholipids of animal or vegetable origin, are used as said emulsifiers. Purified lecithins, such as soy lecithin or egg lecithin or partial fractions obtained therefrom, are preferably employed. The phospholipid content in the emulsion to be employed according to the invention is preferably from 0.4 to 2.0% by weight, preferably from 0.6 to 1.5% by weight, respectively based on the total weight of the emulsion.

Further, co-emulsifiers may be employed, such as the alkali salts of long-chain fatty acids (such as sodium stearate, sodium oleate etc.), or, as sole co-emulsifiers or in combination with others, cholesterol or cholesterol esters (e.g., cholesterol acetate). If alkali salts of long-chain fatty acids are used as co-emulsifiers, and also in the case of using cholesterol or cholesterol esters alone or in combination with other co-emulsifiers, their concentration is from 0.01% by weight to 0.1% by weight, preferably from 0.02 to 0.04% by weight, respectively based on the total weight of the emulsion.

Especially if it contains polyunsaturated fatty acids, such as omega-3 or omega-6 fatty acids, the fat emulsion of the pharmaceutical formulation according to the invention may be enriched with antioxidants, which protect from the formation of undesirable peroxides. Above all, vitamin E (alpha-, beta- or gamma-tocopherol) and vitamin C (e.g., as ascorbyl palmitate) may be used as antioxidants, wherein the vitamins E and C or their isomers or derivatives may be either alone or in combination. Depending on the content of long-chain, especially polyunsaturated, lipids in the formulation according to the invention, the weight proportion of antioxidants is from 0.002 to 0.03% (alpha-tocopherol) or from 0.001 to 0.015% (ascorbyl palmitate), respectively based on the total weight of the emulsion to be employed according to the invention.

In a specific embodiment, the fat emulsion to be employed according to the invention in the pharmaceutical formulation according to the invention additionally contains L-carnitine in an amount of preferably from 0.01 to 0.1% by weight, respectively based on the total weight of the emulsion.

In a further embodiment, the formulation according to the invention may additionally contain the vitamins biotin and/or cobalamine: Their concentrations are from 1 to 10 mg of biotin or from 0.1 to 1 mg of cobalamine per 100 g of lipid fraction of the formulation.

The isotonization of the fat emulsion is preferably effected by means of polyols, such as glycerol, xylitol or sorbitol, which are applied in an amount of preferably from 2 to 3% by weight, respectively based on the total weight of the emulsion. Glycerol serves as a preferred isotonizing agent.

The pharmaceutical agent according to the present invention is administered in a pharmaceutically effective amount. Preferably, the pharmaceutically effective amount, i.e., the amount of triglyceride (A) to be supplied with the pharmaceutical formulation according to the invention in order to avoid the complications of severe, e.g., septic, diseases and their intensive care medical treatment, to alleviate their intensity and shorten their duration, is from 1 to 2 g per kg of body weight per day. The supply is preferably effected continuously over 24 hours/day, but may also be distributed to several portions, wherein an infusion rate of 0.25 g of lipid per kg of body weight per hour should not be exceeded. A medium- to long-term administration for several days is generally required to achieve the effect according to the invention.

The pharmaceutical formulation according to the invention is applied as a component of a completely parenteral or combined parenteral/enteral nutrition, as indicated for severely ill, e.g., septic, intensive care patients showing a complicated course of disease or manifest or imminent neuropathy. The high energy content of the emulsion according to the invention is to be taken into account in the total caloric supply with parenteral or combined enteral/parenteral nutrition. Depending on the embodiment of the fat emulsion according to the invention that is applied, i.e., with or without a proportion of essential fatty acids (omega-3 or omega-6), the latter need not be supplemented additionally. Especially due to the particularly preferred presence of omega-3 fatty acid residues in the emulsion and their anti-inflammatory properties, synergistic effects can be achieved, and the healing process additionally promoted.

The present invention further relates to an isotonic fat emulsion comprising a triglyceride (A) of formula (I):

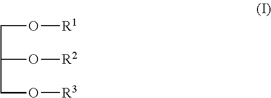

wherein at least one of radicals R¹, R² or R³ is an alkanoyl radical having an odd number of from 5 to 15 carbon atoms, comprising at least one additional triglyceride (B) different from (A) and having at least one fatty acid residue selected from the group consisting of medium-chain fatty acids including caprylic acid, capric acid or lauric acid, long-chain saturated fatty acids including myristic acid, palmitic acid or stearic acid, monounsaturated fatty acids including palmitoleic acid or oleic acid, and polyunsaturated fatty acids of omega-3 and omega-6 type including eicosapentaenic acid, docosahexaenic acid, linolic acid and gamma-linolenic acid.

Further preferred embodiments of the isotonic fat emulsion according to the invention correspond to the fat emulsion to be employed according to the invention in the pharmaceutical formulation according to the invention.

The present invention further relates to the use of the isotonic fat emulsion according to the invention as a dietary product. For this purpose, the isotonic fat emulsion is preferably used for enteral nutrition.

The present invention further relates to a dietary product comprising at least one triglyceride (A) of formula (I). Preferably, the dietary product comprises the isotonic fat emulsion according to the invention.

The present invention further relates to the use of the isotonic fat emulsion according to the invention or of a fat emulsion comprising at least one triglyceride (A) of formula (I) or of the pharmaceutical formulation according to the invention for artificially feeding septic intensive care patients and/or for parenteral nutrition.

The invention further relates to a medicament comprising at least one triglyceride (A) of formula (I):

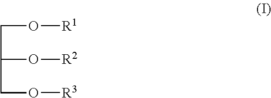

wherein at least one of radicals R¹, R² or R³ is independently an alkanoyl radical having an odd number of from 5 to 15 carbon atoms, and at least one other triglyceride (B) different from (A). Preferably, the medicament comprises the isotonic fat emulsion according to the invention.

For the preparation of the pharmaceutical formulation according to the invention and of the isotonic fat emulsion according to the invention as well as of the medicament according to the invention, the lipophilic components 1 to 9 as stated in the following Table (as required depending on the preparation example) are roughly mixed and dispersed by means of an Ultra-Turrax homogenizer. Subsequently, the hydrophilic components 10 to 13 are added, using sodium oleate or stearate and NaOH as aqueous solutions, and the pH of this initial mixture is adjusted to a value of from 8.0 to 9.0 using the latter mentioned components. The actual homogenization of the mixture is subsequently effected in a high-pressure homogenizer under pressures of about 400 kg/cm². The finished emulsion is filled into suitable glass or plastic containers and heat-sterilized by the methods usually applied for parenteral preparations. What results is a sterile, pyrogen-free and stable fat emulsion having a mean particle size of less than 0.5 μm and a shelf life of at least 24 months at room temperature.

### EXAMPLES

| | | Ex. 1 | | Ex. 2 | | Ex. 3 | | Ex. 4 | | Ex. 5 | | Ex. 6 | | Ex. 7 | |
|---|---|---|---|---|---|---|---|---|---|---|---|---|---|---|---|
| 1 | Triheptanoin | 800 | g | 800 | g | 1400 | g | 1000 | g | 600 | g | — | | 1500 | g |
| 2 | Medium-chain | — | | — | | — | | 400 | g | — | | — | | — | |
| | triglycerides¹⁾ | | | | | | | | | | | | | | |
| 3 | Soybean oil | 200 | g | — | | — | | 300 | g | — | | — | | 1000 | g |
| 4 | Safflower oil | — | | — | | 300 | g | — | | 200 | g | — | | — | |
| 5 | Fish oil concentrate | — | | 200 | g | 300 | g | 300 | g | 200 | g | — | | 500 | g |
| 6 | Structured lipid²⁾ | — | | — | | — | | — | | — | | 1000 | g | — | |
| 7 | Phospholipids | 80 | g | 80 | g | 120 | g | 120 | g | 80 | g | 80 | g | 150 | g |
| 8 | α-Tocopherol | 500 | mg | 500 | mg | 1500 | mg | 1500 | mg | 1000 | mg | 50 | mg | 2000 | mg |
| 9 | Ascorbyl palmitate | 200 | mg | 200 | mg | 600 | mg | 300 | mg | 400 | mg | 200 | mg | 1000 | mg |
| 10 | Sodium oleate | 3.0 | g | 3.0 | g | 3.0 | g | 3.0 | g | 3.0 | g | 3.0 | g | 3.0 | g |

| | | | | | | | | |
|---|---|---|---|---|---|---|---|---|
| 11 | Glycerol | 25 | 25 | 22.5 | 22.5 | 25 | 25 | 20 |
| 12 | NaOH | ad pH | ad pH | ad pH | ad pH | ad pH | ad pH | ad pH |
| | | 8-9 | 8-9 | 8-9 | 8-9 | 8-9 | 8-9 | 8-9 |
| 13 | Water for injection | ad 10 | ad 10 | ad 10 | ad 10 | ad 10 | ad 10 | ad 10 |
| | | liters | liters | liters | liters | liters | liters | liters |

¹⁾For the proportion of medium-chain triglycerides (caprylic acid/caprinic add triglycerides), a commercially available mixture (Miglyol 812, Sasol Germany GmbH, Witten, Germany) was used.
²⁾As a structured lipid, triglyceride (A) consisting of glycerol esterified with heptanoic acid in positions Sn-1 and Sn-3 and eicosapentaenic acid (EPA; C20:5 omega-3) in position sn-2 of the triglyceride was employed

1-17. (canceled)

18. A method of treating critical illness myopathy or critical illness polyneuropathy in a patient, the method comprising:
applying a pharmaceutical formulation comprising a fat emulsion to a patient, wherein the fat emulsion contains at least one triglyceride (A), wherein said triglyceride (A) is triheptanoin, wherein the fat emulsion further comprises at least one additional triglyceride (B) different from (A) and having at least one fatty acid residue selected from the group consisting of medium-chain fatty acids including caprylic acid, capric acid or lauric acid, long-chain saturated fatty acids including myristic acid, palmitic acid or stearic acid, monounsaturated fatty acids including palmitoleic acid or oleic acid, and polyunsaturated fatty acids of omega-3 and omega-6 type including eicosapentaenoic acid, docosahexaenoic acid, linoleic acid and gamma-linolenic acid, further wherein said triglyceride (A) is present in an amount ranging from 50% by weight to 80% by weight of all triglycerides in the fat emulsion, wherein the pharmaceutical formulation is delivered enterally, parenterally, or a combination thereof.

19. The method of claim 18, wherein said triglyceride (A) is in the form of a randomized or chemically defined structured triglyceride.

20. The method of claim 18, wherein the amount of triglycerides is from 5% by weight to 30% by weight, based on the total weight of pharmaceutical formulation.

21. The method of claim 20, wherein the amount of triglycerides is from 10% by weight to 20% by weight, based on the total weight of the pharmaceutical formulation.

22. The method of claim 18, wherein the amount of triglyceride (A) is from 60% by weight to 70% by weight, based on the total weight of all triglycerides in the emulsion.

23. A method of artificially feeding a patient, the method comprising:
applying a pharmaceutical formulation comprising a fat emulsion to a patient, wherein the fat emulsion contains at least one triglyceride (A), wherein said triglyceride (A) is triheptanoin, wherein the fat emulsion further comprises at least one additional triglyceride (B) different from (A) and having at least one fatty acid residue selected from the group consisting of medium-chain fatty acids including caprylic acid, capric acid or lauric acid, long-chain saturated fatty acids including myristic acid, palmitic acid or stearic acid, monounsaturated fatty acids including palmitoleic acid or oleic acid, and polyunsaturated fatty acids of omega-3 and omega-6 type including eicosapentaenoic acid, docosahexaenoic acid, linoleic acid and gamma-linolenic acid, further wherein said triglyceride (A) is present in an amount ranging from 50% by weight to 80% by weight of all triglycerides in the fat emulsion, wherein the pharmaceutical formulation is delivered enterally, parenterally, or a combination thereof.

24. The method of claim 23, wherein said triglyceride (A) is in the form of a randomized or chemically defined structured triglyceride.

25. The method of claim 23, wherein the amount of triglycerides is from 5% by weight to 30% by weight, based on the total weight of pharmaceutical formulation.

26. The method of claim 25, wherein the amount of triglycerides is from 10% by weight to 20% by weight, based on the total weight of the pharmaceutical formulation.

27. The method of claim 23, wherein the amount of triglyceride (A) is from 60% by weight to 70% by weight, based on the total weight of all triglycerides in the emulsion.
